(19) United States
(12) Patent Application Publication
(10) Pub. No.: US 2003/0148354 A1
(43) Pub. Date: Aug. 7, 2003

(54) Devices and methods for isolating and detecting specific substances in complex matrices

(75) Inventor: Virginia Gordon, Huntington Beach, CA (US)

Correspondence Address: Robert D. Buyan, Stout, Uxa, Buyan & Mullins, LLP, Suite 300, 4 Venture, Irvine, CA, 92618, US

(73) Assignee: Saftest, Inc., Phoenix, AZ (US)

(21) Appl. No.: 10/325,505

(22) Filed: Dec. 20, 2002

Related U.S. Application Data
(63) Continuation-in-part of application No. 09/183,157, filed on Oct. 30, 1998, now abandoned.
(63) Continuation-in-part of application No. 08/723,636, filed on Oct. 2, 1996, now Pat. No. 5,958,714.
(60) Provisional application No. 60/342,425, filed on Dec. 20, 2001.

(51) Int. Cl. C12Q 1/68; C12M 1/34; G01N 33/558

(52) U.S. Cl. 435/6; 435/287.2; 436/514

(57) ABSTRACT

Methods and systems for determining the presence of one or more analytes in various types of matricies, such as foods, oils, biological fluids, etc. Membranes are used to remove extraneous matter (e.g., solid particles) and/or to bind specific interferents or analytes. Specific assays are provided for determining citric acid, total fat (or % fat), free fatty acids, protein content and specific nucleic acid content indicative of the presence of DNA and/or RNA of certain microbes (e.g., pathogens) or genetically altered substances (e.g., genetically engineered grain).

### RELATED APPLICATIONS

This application claims priority to U.S. Provisional Application No. 60/342,425 filed on Dec. 20, 2001, the entirety of which is expressly incorporated herein by reference. Additionally, this application is a continuation in part of copending U.S. patent application Ser. No. 09/183,157 filed on Oct. 30, 1998 which is a continuation in part of U.S. patent application Ser. No. 08/723,636 filed on Oct. 2, 1996, now U.S. Pat. No. 5,958,714 filed on Oct. 2, 1996. Copending U.S. patent application Ser. No. 09/183,157 and issued U.S. Pat. No. 5,958,714 are also expressly incorporated herein by reference.

### FIELD OF THE INVENTION

The present invention pertains generally to methods and apparatus for analytical chemistry, and more particularly to test kits and methods for qualitatively or quantitatively determining one or more analytes present within a sample or matrix.

### BACKGROUND OF THE INVENTION

It is routinely desirable to test for the presence of specific analytes in substances which are intended for human consumption or application to the human body (e.g., foods, beverages, cosmetics, toiletries, topical solutions, contact lens solutions, pharmaceutical preparations, etc.) to confirm that such substances are fresh (i.e., not degraded), pure and free of contamination. Additionally, it is often desirable to test for the presence of specific analytes in samples of biological fluids (e.g., blood, plasma, serum, urine, saliva, bile, lymph, etc.) which have been extracted from the human body.

However, the analytical techniques which have heretofore been utilized to quantitatively or qualitatively test for specific analytes in complex matrices are often problematic, due to the fact that such substances may contain many diverse physical and/or chemical species, some or all of which may interfere with the intended analysis. Thus, it is frequently necessary for the test substance to be subjected to extensive sample preparation steps, in order to isolate and/or concentrate the particular analyte(s) of interest, prior to actually proceeding with analytical determination of the desired analyte(s). Moreover, in instances where the test substance is a solid material (e.g., food) it is often necessary to chop or grind the solid material into particles, and to extract the desired analyte(s) from such particles by adding one or more liquid digestants, solvents or other fluids to form a slurry or suspension, and thereafter performing a “clean up” of the slurry or suspension by filtration or centrifugation to separate the analyte containing liquid from the extraneous solid matter. In instances where multiple analytes are to be determined, it is often necessary to perform several separate, time consuming, analytical procedures (e.g., gas chromatography (GC), high performance liquid chromatography (HPLC) or other analytical chemistry procedures) on aliquots or extracts of the test substance, in order to generate the desired multiple analyte data.

Thus, the traditional methods for determining the presence of, or detecting specific analyte(s) in complex matrices (e.g., substances which contain matter other than the desired analyze(s)) can be quite time consuming, skill intensive and expensive.

It is frequently desirable to detect or quantify, in foods, one or more particular analyte(s) which are indicative of the freshness or quality of the food. In routine quality control testing of foods, it is common practice to test for the presence of various contaminates, additives, degradation products, and/or chemical markers of microbial infestation (e.g., bacterial endotoxins, mycotoxins, etc.). However, the current methods by which such quality control testing of food is accomplished are typically either: a) complex and skill-intensive analytical chemistry procedures or b) highly subjective and qualitative sensory evaluations (e.g., smell test, taste test, appearance, etc.).

The quantities of certain food additives may be subject to governmental regulation, especially in formulations wherein synthetic additives are being utilized. Thus, in such situations, it is typically desirable to perform chemical analyses as means of determining the minimum amount(s) of particular antioxidant additives which must be added to a particular formulation to provide the desired effect and/or to identify non-regulated natural alternatives to governmental regulated synthetic additive. Thus, the detection and/or analysis of certain additives in foods and other formulations is often carried out for various product/formulation development or research purposes, as well as for quality control testing of the freshness and wholesomeness of the food or other product.

Also, bacterial or microbial contamination of foods and other substances is an ongoing problem in a number of industries. In many instances, microbiological culture techniques are used to test for the presence of undesirable microbial contaminants in foods and other substances. These microbiological culture techniques often take several days to complete and are subject to human error. While PCR and other genetic techniques have been developed to quickly test for the presence of specific microbial DNA or RNA, the use of those techniques can be problematic when the suspected microbial contamination is contained within a food or other complex matrix. Thus, there remains a need for the development of new techniques for rapidly separating or isolating microbial DNA or RNA from a complex matrix such as a food and to thereafter detect the presence of such microbial DNA or RNA without the need for time consuming and laborious microbiological culturing.

In view of the foregoing problems and because the previously-known analytical methods for determining specific analytes in relatively complex matrices (e.g., foods, biological fluids, etc.) may be too complex or too skill-intensive for untrained personnel, there exists a need in the art for the development of simple test kits capable of rapidly and reproducible determining the presence and/or concentrations of certain analytes or the presence of certain nucleic acid sequences in complex matrices, so that relatively untrained-personnel may perform such determinations in a reliable, cost effective manner.

Some of the shortcomings of the prior art were overcome by the inventions described in Applicant's copending U.S. patent application Ser. No. 09/183,157 and previously issued U.S. Pat. Nos. 5,958,714 and 6,489,123, the entireties of which are expressly incorporated herein by reference.

### SUMMARY OF THE INVENTION

The present invention provides methods and systems (e.g., test kits) for qualitative and/or quantitative determination of one or more analytes present within a matrix that contains matter other than the analyte (e.g., solids, particulate matter, matter or substances that will interfere with the analysis, etc.). These methods and apparatus are useable to detect or quantify specific analytes present in complex matrices such as foods, cosmetics or biologicals, organ/tissue homogenates, industrial waste, sewage, industrial fluids, microbiological or pharmaceutical incubator slurries, etc. to determine the quality, degradation, age, abuse, contamination, nutritional value, purity and other characteristics of the matrices.

In accordance with this invention, there is provided a method and system (e.g., test kit) for determining the presence of a single analyte. This system comprises; a) a sample receiving vessel, b) a membrane and c) a reagent-containing well. The test sample is initially prepared (e.g., chopped or ground if a solid) and is deposited in the sample-receiving vessel along with any desired diluent, digestion solution (e.g., enzymes), chelators, or chemical modifiers (e.g., antioxidants). The prepared sample is then permitted to drain from the sample-receiving vessel, through the membrane. The type of membrane utilized in each embodiment will be selected based on the type and quantity of matter which is desired to be excluded from the prepared sample matter prior to analysis. In many applications, this initial membrane will be formed of microporous film having pores which are sized to present large particles of solid matter, proteins and other unwanted matter from passing therethrough, but which will allow a filtrate containing the desired analyte to drain into the reagent-containing well. When drained into reagent-containing well, the analyte contained within the filtrate will react with the reagent in a manner which will permit the presence or quantity of analyte to be determined. In many instances, the analyte-reagent reaction will be a color forming reaction such that a visual determination may be made as to whether, or to what degree the desired analyte is present. In other instances, it may be desirable to utilize an analytical instrument to determine the quantity of analyte present in the analyte present in the analyte-reagent solution. Examples of specific apparatus that may be used to support the membranes, facilitate flow of the sample/filtrate through the membranes and collection of the filtrate(s) and eluants for subsequent analysis are found in copending U.S. patent application Ser. No. 09/183,157 and previously issued U.S. Pat. Nos. 5,958,714 and 6,489,123, the entireties of which are expressly incorporated herein by reference.

Further in accordance with this invention, a method or system of the above-described character may be adapted for determination of two or more analytes by the addition of one or more additional membranes in series with the first membrane. Each of these additional membranes is operative to capture and hold at least one analyte, while allowing a filtrate containing one or more other analyte(s) to pass therethrough. Each of these additional membranes may subsequently be exposed to a wash or flush solution such that one or more eluants containing each of the additional analytes may be obtained. Each such eluant may subsequently be combined with a reagent to provide an eluant-reagent admixture from which at least one analyte may be determined. In this manner, the present invention is adaptable for the qualitative or quantitative determination of two or more analytes from a single sample.

Further in accordance with this invention, in situations where one or more analytes is/are present in a matrix at low concentrations (e.g., concentrations that are below the detection limit of the intended analytical test) the analyte may be captured on a membrane and may be subsequently eluted from that membrane with a volume of eluent that is substantially smaller than the volume of the original sample, thereby providing an analyte/eluant admixture wherein the concentration of the analyte is sufficiently high to permit its detection by the intended analytical method. The starting concentration of the analyte in the original sample may then be determined by calculation based on the known volume of the original sample and the known volume of the eluant that was used to elute the analyte from the membrane.

Further in accordance with this invention, there are provided methods and systems of the foregoing character wherein a membrane is used to remove a positive or negative interferant from the sample to permit an analyte to be analyzed or detected by chemical or biochemical methods without interference. One particular embodiment of this invention wherein an analyte is removed comprises a method and system wherein free fatty acids (FFA) are present in a sample (e.g., a food or oil) along with one or more inorganic acids. The analytical method intended to be used to detect or to quantitate the presence of FFA will also detect the presence of inorganic acids. Therefore it is desired to remove the inorganic acid(s) from the sample prior to analysis for the FFA. To accomplish this, the sample is passed through at least one negatively charged membrane that captures inorganic acids but allows a filtrate containg any FFA's present in the sample to pass therethrough. The FFA containing filtrate is then subjected to the analytical test for FFA's and an accurate quantitative or qualitative determination of FFA's is then obtained. In some situations it is additionally desired to qualitatively or quantitatively analyze the inorganic acid that was present in the sample. In such situations, an eluant that releases the inorganic acid from the negatively charged membrane is used to elute the inorganic acid from the membrane on which it was captured, thereby providing an inorganic acid/eluant admixture from which the inorganic acid may be quantitatively or qualitatively analyzed. In some situations it may be additionally desirable to desperate specific types of inorganic acids present in the sample and to analyze for one or both of those types of inorganic acids. Accordingly, in such instances, the sample may be passed through a plurality of membranes, each of which has a binding affinity for a different type of inorganic acid, before the filtrate is analyzed for FFA. In this regard, a first membrane may be impregnated or coated with a substance which carries a sufficient negative charge to bind weak inorganic acids (e.g., acetic acid) and a second membrane may be impregnated or coated with a substance which carries a sufficient negative charge to bind stronger inorganic acids (e.g., citric acid). The weak and strong inorganic acids that become bound to these membranes may then be separately eluted and analyzed, if desired. In other instances, it may be desirable to perform an enzymatic analysis for a particular analyte contained in a sample but the presence of metals in the sample may interfere with such enzymatic analysis. In such instances, the sample may be passed through an anionic membrane which will bind and hold metals present in the sample and the desired enzymatic analysis may then be performed on the metal free filtrate without interference from the previously present metals.

Still further in accordance with this invention, there are provided methods and systems wherein the sample is passed through a membrane (e.g., a membrane that is impregnated or coated with specific antibodies) which binds certain amino acid sequences. The particular amino acid sequence may be selected on the basis of its known presence in the nucleic acid (e.g., DNA or RNA) of a particular organism or microbe (e.g., bacteria, virus, parasite, spore, prion, etc.), a genetically modified substance or a protein, that may be present in the sample. The bound nucleic acid(s), genetically modified substance(s) or protein(s) are then eluted or released from the membrane and subjected to an analytical or detection technique, such as amplification and PCR, whereby a quantitative or qualitative determination of that nucleic acid or protein is made. This aspect of the invention is useable to determine the presence or concentration of certain pathogenic or deleterious microbes, toxic or deleterious proteins, or the presence of a prohibited or regulated substance (e.g., genetically modified plant substances or grain) in a food, beverage, water, medicine, cosmetic or other sample.

Still further in accordance with this invention, there are provided methods and systems wherein a sample is passed through a pre-weighed membrane which has a selective affinity to bind a certain substance. The membrane with the substance bound thereto is then reweighed to determine the weight of the substance that was present in the sample. In this regard, a food or beverage sample may be passed through a membrane that has a specific binding affinity for proteins. Thereafter the membrane (with the protein bound thereto) may be weighed and the weight of the protein removed from the sample may be calculated. On this basis, one may also calculate the % protein present in the sample. Alternatively, the protein may be eluded from the membrane and analyzed as described herein.

### BRIEF DESCRIPTION OF THE DRAWINGS

FIG. 1 is a schematic diagram of a single membrane device useable with some of the methods and systems of the present invention.

FIG. 2 is a schematic diagram of a plural membrane useable with some of the methods and systems of the present invention.

FIG. 3 is a table listing specific filtration and capture membranes that may be used in the present invention.

FIG. 4 is a table listing specific detection reagents that may be used for detection or analysis of analytes in the present invention.

FIG. 5 is a table listing specific test methods and systems and specifying the analytes, typical matricies in which the analyte is contained, specific membranes (cross-referenced to FIG. 3) and the specific detection reagents (cross-referenced to FIG. 4) useable in each test method and system.

### DETAILED DESCRIPTION

The following detailed description and the figures to which it refers are not intended to describe all possible embodiments and examples of the invention. Rather, this detailed description and the accompanying figures are directed to certain illustrative embodiments and examples of the invention only and does not limit the scope of the invention in any way.

Methods and Systems of the Present Invention:

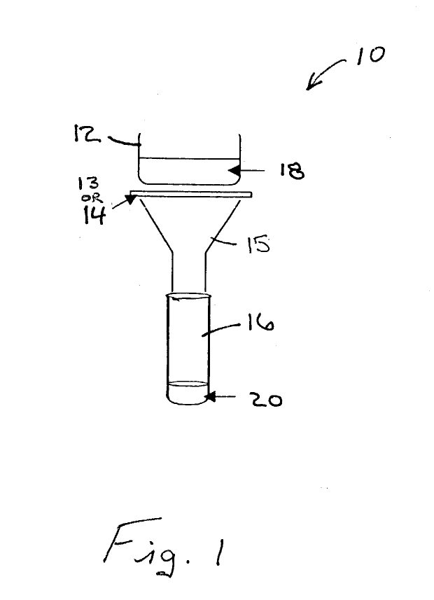

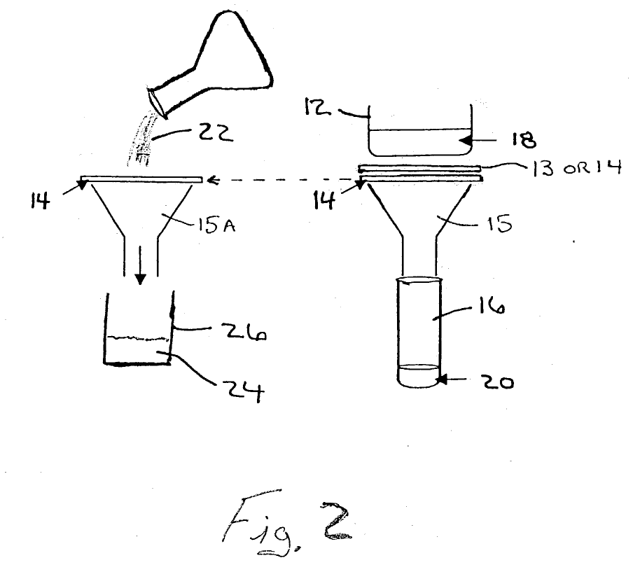

The present invention includes a number of specific methods and systems (e.g., combinations of membranes, eluants and reagents; test kits) that may be used to obtain quantitative or qualitative determinations of specific analytes in foods, oils and other matrices. The methods and systems may be used in conjunction with the devices described in copending U.S. patent application Ser. No. 09/183,157 and previously issued U.S. Pat. Nos. 5,958,714 and 6,489,123, the entireties of which are expressly incorporated herein by reference. Certain embodiments of these devices are commercially available as the Saftest™ Membrane Unit and the Saftest™ Filtration Unit from Saftest, Inc., 3550 North Central, Suite 1400, Phoenix, Ariz. 85012. FIGS. 1 and 2 show, in schematic fashion, examples of devices used in conjunction with the methods and systems of this invention.

Specifically, FIG. 1 shows a single membrane device 10. This single membrane device 10 comprises a sample well 12, a membrane support 15, and a filtrate collection well 16. In embodiments where the sample 18 comprises matrix that includes a liquid phase wherein the analyte as well as extraneous matter (e.g., solid particles or large molecular weight compounds) a filtration membrane 13 having pores that are small enough to prevent passage therethrough of the extraneous matter but large enough to permit passage therethrough of the analyte-containg liquid phase is positioned on the membrane support. The sample 18 then passes from the sample well 12 and through the filtration membrane 13, whereby the extraneous matter is retained above the membrane and a filtrate 16 containing the analyte passes through the filtration membrane 13 and into the filtrate collection well 16. A desired analytical or detection technique may then be used to quantitatively or qualitatively determine the analyte in the filtrate 20. In some instances, such analysis will require one or more reagents to be mixed with the analyte-containing filtrate 20. In other instances, the neat filtrate 20 may be used for the analysis (e.g., examined microscopically, placed in an analytical instrument such as a spectrophotometer or chromatograph or applied to an indicator (e.g., pH paper, paper or dip sticks which indicate the presence of the analyte, etc.) In other embodiments, the sample 18 may be substantially free of extraneous matter that must be removed by a filtration membrane 13 (e.g., a clean oil or liquid solution) but, instead, the sample 18 may contain two analytes that must be separated or some interferant that will interfere with analysis for the analyte and must therefore be separated from the analyte prior to analysis. In these embodiments, a capture membrane 14 will be mounted on the membrane support rather than a filtration membrane 13. This capture membrane 14 may be selected so as to capture (e.g., chemically bond to or otherwise hold) a first analyte while allowing a second analyte to pass therethrough in the filtrate 20. The first analyte may subsequently be eluted (e.g., released) from the capture membrane and determined separately and the first analyte contained in the filtrate 20 may also be determined. The capture membrane may also be used to capture an interferant while allowing a filtrate containg the analyte to pass therethrough or vice versa.

FIG. 2 shows, in schematic fashion, a two membrane device. Here, the top membrane is either a filtration membrane 13 (for samples 18 which contain extraneous matter that must be filtered out) or a capture membrane 14 (for samples that contain multiple analytes or interferants). The bottom membrane is a capture membrane 14. The sample 18 passes through the top membrane which removes extraneous matter or captures a first analyte or interferant. The filtrate that has passed through the top membrane then passes through the bottom membrane which captures an analyte or interferant and the filtrate 20 that has passed through both membranes then collects in the filtrate well 20. An analyte contained in the final filtrate 20 may be determined as described above. If one or both of the membranes have been used to capture another analyte(s), such other analyte(s) may be eluted from the membrane(s) and determined separately. In the example of FIG. 2, it shows that the bottom capture membrane 14 is transferred to a second membrane support 15a. An eluant 22 is then passed through the capture membrane 14 so as to elute (e.g., release) the analyte from that membrane 14. An eluant/analyte admixture 24 is then collected in a collection well 26. The second analyte may then be quantitatively or qualitatively determined from the eluant/analyte admixture. As summarized above, in some embodiments, it may not be necessary to elute the second analyte from the membrane. Rather, the membrane may contain an indicator that changes to indicate the presence of the analyte thereon or the membrane may be weighed to determine the weight of the analyte contained thereon.

As explained in incorporated U.S. Pat. No. 6,489,123, and copending parent application Ser. No. 09/183,157 more than two, and virtually any number, of membranes 13, 14 may be used to capture and optionally analyze virtually any number of analytes or inerferants.

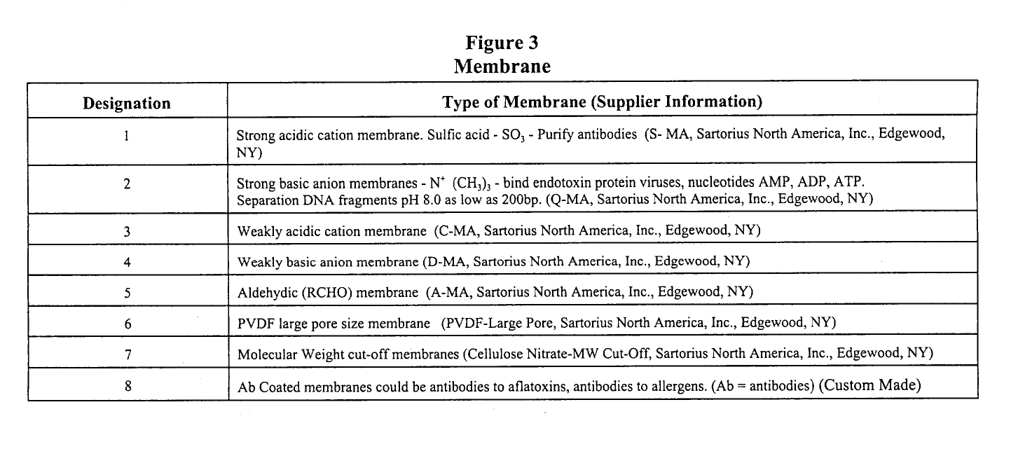

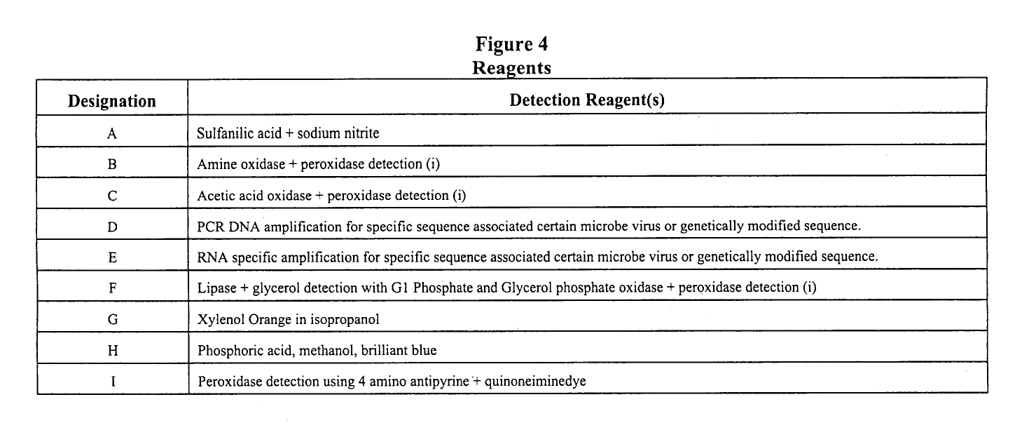

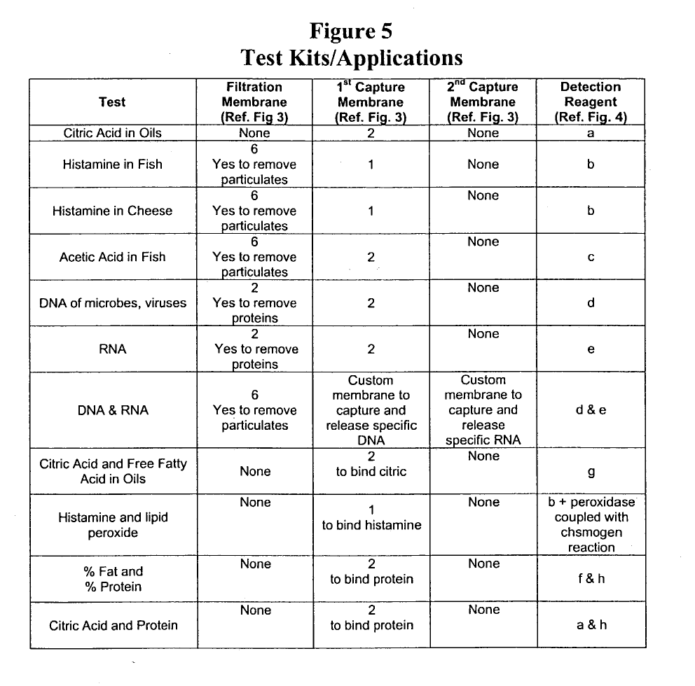

Examples of the filtration membranes 13, capture membranes 14 and reagents useable for specific embodiments of the present invention are shown in the tables of FIGS. 3, 4 and 5. Specific embodiments of the present invention include the following:

1. A method and system for citric acid and free fatty acid determinations in any sample, for example, an oil. The sample (e.g., oil) is passed through a positively charged anionic membrane for capture of the citric acid from the oil and detection of free fatty acids in the filtrate. The citric acid that has become bound to the positively charged anionic membrane is then eluted or released from the membrane using a high salt solution (e.g., 0.5 M NaCl in water) as the eluant. The eluant/citric acid admixture is then combined with sulfanilic acid hydrochloride with a nitrite activator (e.g., 0.2% sulfanilic acid and 5% sodium nitrite). This results in a color reaction indicative of the presence of citric acid. In addition to oils, this citric acid/free fatty acid system can be used for determinations in various other matrices including food. In foods which contain encapsulated lipids, the food may be soluablized such that the lipids are dissolved in a liquid phase. A first membrane may be used to remove solid extraneous matter. The liquid, lipid-containing filtrate is then passed through the capture membrane such that the citric acid becomes bound to the capture membrane. The free fatty acids are then measured in the filtrate that passes through the capture membrane. The citric acid is then released from the capture membrane by elution with a salt solution as described above. The eluant/citric acid admixture may then be contained in a second vessel and the presence and/or amount of citric acid may be analyzed as described above.

2. A method and system for determining acetic acid and free fatty acid can be used for determinations in other food matrices and encapsulated lipids in foods where the food is solubilized. In the same manner as the citric acid assay described above, a first filtration membrane is used to remove particles and other solid matter. The acetic acid containg bound to the capture membrane, the free fatty acids measured in the effluent and then the acetic acid released from the capture membrane with high salt solution into a second vessel and quantitated.

4. Test kit for alkenal acid determinations in oil. A first filtration membrane and oil in food using a particulate removing filtration membrane and then a methyl indole or methylphenyl indole detection system with a very strong acid such as methane sulfonic acid.

5. Test kit for prediction of oxidative degradation of seafood using a particulate removing membrane and then malonaldehyde as a detector to quantitate indolic compounds formed in the degradation of shrimp.

6. Any of the above kits s to be used in conjunction with a second test for malonaldehyde utilizing a methyl indole reagent with weak acid such as small amount of HCl.

7. Any of the above kits to be used with a second test for lipid peroxides in the eluant using an iron catalyzed electron transfer to xylenol orange.

8. Any of the above kits to be used with a second test for Free Fatty acids using an alcoholic indicator such as isopropanol/xylenol orange.

9. A test kit for protein determination on filtered and unfiltered oils in conjunction with the citric acid determination by using one membrane to bind citric acid and one to bind protein and eluting each membrane separately and detecting the analyte.

10. A test kit for protein determination on refined oil concentrating the protein on a protein binding membrane by passing 1 to 200 ml of oil through the membrane and eluting the protein off into another tube using a salt solution in 1 ml.

11. A test kit for protein determination in meals by digesting the meal with phosphoric or another strong acid and using a particulate removing membrane to remove debris and then testing the filtrate for protein.

12. A test kit for protein determination on tallows or greases by using a membrane to bind protein and eluting the membrane and detecting the analyte.

13. A test method and kit for determination of polymerized and non-polymerized oils in cooking fats and oils in conjunction with the alkenal determinations on filtrate to determine frying oil quality and oil. Quality in fried foods using a molecular weight cutoff membrane to capture the polymerized lipids and then release them to measure triglyceride content.

14. A test method and kit to determine oxidation of beverages and determination efficacy of certain additives and/or stabilizers on oxidation using the alkenal test. The beverage, carbonated or not, is separated through protein binding membrane and the filtrate tested with methylindolis solution with sulfonic acid.

15. A test for rapid determination of the quality of cooking oils and fats by testing for lipid peroxides using a peroxidase and iron catalyzed reagent and complexed to xylenol orange and alkenals using the methylindole reagent with a very strong acid added This test can be used on beer or beverages and predict quality and shelf life of beverages.

16. A test method and kit to detect specific microbes or viruses in foods or tissues by emulsifying the food and releasing the nucleic acids using surfactants or osmotic changes to lyse the membranes and cells and using a particulate binding membrane followed by a nucleic acid binding membrane. The DNA is released and then amplification of a sequence specific to the target organism to detect its presence performed.

17. A test method and kit to detect aflatoxins in foods or tissues by emulsifying the food and releasing-the aflatoxins using surfactants or osmotic changes to lyse the membranes and cells and after filtering out particulates using a second membrane coated with an antibody specific to multiple or particular aflatoxins. The aflatoxins are released and then detected using peroxidase conjugated antibodies

18. A test method and kit to detect specific live microbes or viruses in foods or tissues by emulsifying the food and releasing the nucleic acids using surfactants or osmotic changes to lyse the membranes and cells and using a particulate binding membrane followed by a ribonucleic acid binding membrane. The RNA is released and then amplification of a sequence specific to the target organism to detect its presence performed.

Detailed Examples Of Specific Embodiments Of The Present Invention

The following examples demonstrate methods of detecting various analytes contained in samples, in accordance with the invention disclosed hereinabove. The analytes may be removed from a sample using a device or system incorporating one or more membranes for filtering the sample, such as devices and systems disclosed in commonly owned PCT International Patent Publication No. WO 99/20396 and U.S. Pat. No. 6,489,132, and the publicly available SafTest™ Filtration Unit available from Saftest, Inc. (Phoenix, Ariz.). PCT International Patent Publication No. WO 99/20396 and U.S. Pat. No. 6,489,132 are expressly incorporated herein by reference.

### EXAMPLE 1

### Separation and Determination of Free Fatty Acid (FFA) and Citric Acid in an Oil Sample

This example demonstrates free fatty acids contained in an oil sample. The oil sample also contains citric acid. It is desirable to separate the citric acid from the sample prior to assay of the FFA content as the presence of inorganic acids such as citric

A 1 mL sample of soybean oil is applied to a membrane of a filtering device. The membrane is a strongly basic anionic membrane, such as the Q membrane adsorber membrane with quaternary ammonium groups (Q-MA membrane) publicly available from Sartorius (Sartorius North America, Inc., Edgewood, N.Y.). As the sample is applied to the membrane, the citric acid is retained by the membrane, and the remaining oil containing free fatty acids is collected in a container.

The membrane containing the citric acid is removed from the container and is washed with 1 mL of 0.5 M NaCl in water. The eluant is collected in a second container. One mL of the eluant containing citric acid is mixed with 0.3 mL of a reagent containing 0.2% sulfanilic acid and 5% sodium nitrite. The reaction occurs for about 30 minutes an elevated temperature (approximately 42-45° C.). The presence of citric acid in the sample results in a yellow color which can be measured by examining the reaction mixture with a spectrometer at 420 nm, and comparing the calculation to one or more standards. A test kit suitable for performing this citric acid assay is commercially available under the name CitriSafe™ from Saftest, Inc. (Phoenix, Ariz.). The CitriSafe™ test kit is generally described in Appendix A to this patent application.

The amount of free fatty acids originally present in the soybean oil is determined by measuring the acidity of the oil after the removal of the citric acid using the methodology described in incorporated parent application Ser. No. 09/183,157 and commercially available as a test kit under the name FASafe™ from Saftest, Inc. (Phoenix, Ariz.).

The CitriSafe™ and FASafe™ test kits are useable in conjunction with devices described in copending U.S. patent application Ser. No. 09/183,157 and previously issued U.S. Pat. Nos. 5,958,714 and 6,489,123, the entireties of which are expressly incorporated herein by reference. Certain embodiments of these devices are commercially available as the Saftest™ Membrane Unit and the Saftest™ Filtration Unit or Saftest™ Work Station, from Saftest, Inc.

### EXAMPLE 2

### Removal of Interfering Inorganic Acids and Determination of Free Fatty Acid (FFA) and Acetic Acid in a Food Sample

This example demonstrates the separation of inorganic acids from free fatty acids in a food sample and the subsequent determination of acetic acid and free fatty acid content of that food sample.

A 5 gram sample of mackerel is solubilized to create a slurry. The slurry is heated to approximately 40-45° C. and filtered to remove particulates from the slurry. Two (2) mL of the filtered slurry is applied to a membrane structure of a filtering device. The membrane structure includes two stacked membranes one disposed on top of the other. The upper membrane is a weakly basic membrane, such as the D membrane adsorber with diethylamine groups (the MA-D membrane), and the lower membrane is a strongly basic membrane, such as the membrane used in Example 1. These membranes are publicly available from Sartorius (Sartorius North America, Inc., Edgewood, N.Y.). As the filtered slurry is applied to the membrane structure, acetic acid, and other weak inorganic acids, are retained by the upper membrane, and citric acid, and other strong inorganic acids are retained in the lower membrane. The remaining slurry containing free fatty acids is collected in a container.

The membrane containing the acetic acid is removed from the container and is washed with 2 mL of 1 M NaCl in water. The eluant is collected in a second container. 100 μl of the eluant containing acetic acid is mixed with 1.0 mL of a reagent containing 0.1% xylenol orange in neutralized isopropanol. The reaction occurs for about 10 minutes at an elevated temperature (approximately 42-45° C.). The presence of acetic acid in the sample is determined by examining the reaction mixture with a spectrometer at 570 nm. The amount of acetic acid present in the sample is determined by comparing the results to one or more standards.

The citric acid is removed from the lower membrane using the procedure disclosed in Example 1.

The amount of free fatty acids originally present in the fish slurry is determined by measuring the acidity of the oil after the removal of the inorganic acids using the FASafe™ publicly available from Saftest, Inc. (Phoenix, Ariz.). The FASafe™ test kit is useable in conjunction with devices described in copending U.S. patent application Ser. No. 09/183,157 and previously issued U.S. Pat. Nos. 5,958,714 and 6,489,123, the entireties of which are expressly incorporated herein by reference. Certain embodiments of these devices are commercially available as the Saftest™ Membrane Unit and the Saftest™ Filtration Unit or Saftest™ Work Station, from Saftest, Inc.

### EXAMPLE 3

### Determination of Total Fat Content and/or Percent Fat

This example demonstrates the determination of fat content or the percent of fat in foods.

Eight ounces of salad dressing is heated and homogenized with stabilized 100% isopropanol to release lipids in the salad dressing that are bound to proteins or held in membranes of items in the salad dressing. The homogenate is prefiltered to remove particulates using a cellulose acetate membrane having a pore size of 0.45 microns. The filtered homogenate is passed through a membrane that binds proteins, such as the polyethersulfone (PES) membrane sold by Sartorius, Inc, and then the filtered homogenate is passed through a membrane that binds surfactants, such as the MA-Q or MA-S membranes from Sartorius. The MA-S membrane has sulfonyl groups on the membrane surface for binding surfactants.

A portion of the filtrate (20 μL) that is free of proteins and surfactants is mixed with 1.0 mL of lipase (Sigma, St. Louis, Mo.) in phosphate buffer to enzymatically cleave the fatty acids from glycerol. The amount of glycerol present in the filtrate is measured enzymatically using a series of enzyme reactions using glycerol kinase and ATP to produce glycerol 1-phosphate and glycerol-1 phosphatase to produce dihydroxyacetone, which is detected with a peroxidase catalyzed reaction with aminoantipyrine to produce a measurable quinoneimine dye. This reaction is complete in 10 minutes at 42° C. By measuring the amount of glycerol present in the filtrate, the total fat content contained in the salad dressing is determined without regard to the specific proportions of the various proportions of free fatty acids.

A test kit for this percent fat assay is commercially available as Percent Fat Kit MSA from Saftest, Inc. (Phoenix, Ariz.) and is described in Appendix B to this patent application. The Percent Fat Kit MSA is useable in conjunction with devices described in copending U.S. patent application Ser. No. 09/183,157 and previously issued U.S. Pat. Nos. 5,958,714 and 6,489,123, the entireties of which are expressly incorporated herein by reference. Certain embodiments of these devices are commercially available as the Saftest™ Membrane Unit and the Saftest™ Filtration Unit or Saftest™ Work Station, from Saftest, Inc.

### EXAMPLE 4

### Determination of Protein Content in Refined Oil

In this example the present invention is used to determine total protein content in a refined oil such as soy bean oil.

A 5 mL sample of refined and genetically modified soy bean oil is heated to approximately 40° C. and is mixed with 5 mL of 100% isopropanol. The warm mixture is applied to a membrane that binds proteins, such as the membrane used in Example 1. The protein in the oil/alcohol mixture binds to the membrane, and the fatty acids contained in the mixture pass into a container.

The protein-containing membrane is moved to another container and is washed to release the protein into the container with 1 mL of buffered, low salt solution (0.05 M NaCl in phosphate buffer at a pH between 7 and 9). One (1) mL of the concentrated filtrate is mixed with 0.3 mL of an indicator solution containing 0.1% brilliant blue (Sigma, St. Louis, Mo.) in 30% methanol, and 0.3% phosphoric acid for two minutes at room temperature (18-25° C.). The presence of protein is qualitatively determined by the presence of a blue color in the mixture. The amount of protein is quantified by comparing the blue color of the mixture to one or more standards, and/or by using a spectrometer at 570 nm.

A test kit for this protein content assay is commercially available as ProteSafe™ from Saftest, Inc. (Phoenix, Ariz.) and is described in Appendix C to this patent application. The ProteSafe™ test kit is useable in conjunction with devices described in copending U.S. patent application Ser. No. 09/183,157 and previously issued U.S. Pat. Nos. 5,958,714 and 6,489,123, the entireties of which are expressly incorporated herein by reference. Certain embodiments of these devices are commercially available as the Saftest™ Membrane Unit and the Saftest™ Filtration Unit or Saftest™ Work Station, from Saftest, Inc.

### EXAMPLE 5

### Determining the Presence of a Particular Microbe in a Food Sample

This example demonstrates methods to identify the presence of one or more microbes, including pathogenic and non-pathogenic bacteria and viruses, in food products. The microbes are detected by binding nucleic acids to one or more membranes, and amplifying the nucleic acids using nucleic acid primers having a desired nucleotide sequence for the microbes.

Ten grams of ground beef is prepared for determination of the presence of ecoli H157. The ground beef is homogenized with a buffered solution, such as phosphate buffer, containing 1-2% sodium dodecyl sulfate (SDS) in a ratio of approximately 1 to 4, of beef to diluent, to disrupt the cellular component and to release nucleic acids contained within the beef. The slurry of homogenized ground beef is applied to a first membrane, such as a polytetraflouroethylene membrane (available from Sartorius), to remove particulates from the ground beef. The filtered slurry is then applied to a second membrane that is configured to bind DNA or RNA. Anionic membranes, such as MA-Q membranes from Sartorius, or membranes having one or more types of nucleic acid binding antibodies, such as MA-A membranes (Sartorius), which has crosslinked antibodies attached to it by glutaraldehyde crosslinking, or the MA-I Iminodiacetic acid membranes (Sartorius), which are reacted with the protein amino groups of the antibodies. The slurry is passed through two additional nucleic acid binding membranes to increase the amount of nucleic acid removed from the homogenate. The filtrate is then discarded. The membranes are washed with 1 M NaCl in water to release the nucleic acids from the membranes into a container. The RNA and DNA are then amplified using polymerase chain reaction (PCR) and one or more nucleic acid primers that have sequences for ecoli H157. PCR methods are conventionally known to persons of ordinary skill in the art, see, for example, Sambrook et al., Molecular Cloning: A Laboratory Manual, 3^{rd} Edition, 2001. The PCR products are labeled by incorporating a fluorescent marker during the amplification steps, and the presence of ecoli H157 is determined by measuring the fluorescence contained in the PCR products.

Although exemplary embodiments of the invention have been shown and described above, many changes, modifications, substitutions, variations and/or additions may be made by those having ordinary skill in the art without necessarily departing from the spirit and scope of this invention. For example, where this patent application has described the performance of steps of a method or procedure in a specific order, it may be possible (or even expedient in certain circumstances) to change the order in which some steps are performed, and it is intended that the particular steps of the method or procedure claims set forth herebelow not be construed as being order-specific unless such order specificity is expressly stated in the claim. Another example is that, although specific membranes and reagents are called out above, various other membranes or reagents or equivalent materials may be used to bring about the same or substantially the same effects as described herein and those other membranes and reagents may also be useable to practice the methods of the present invention. Accordingly, it is intended that all such changes, modifications, substitutions, variations and/or additions be included within the scope of the following claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A system for determining the presence of an analyte in a sample which contains matter other than said analyte, said system comprising:
an analyte capture membrane which is operative to capture an analyte;
an eluant for eluting the analyte from the capture membrane so as to provide an eluant/analyte admixture; and, at least one reagent which is combinable with the eluant/analyte admixture to provide a reagent/eluant/analyte admixture from which said analyte may be determined.

2. A system according to claim 1 further comprising:
a first membrane which is operative to prevent extraneous matter contained in the sample from passing therethrough, while allowing a filtrate containing said analyte to pass therethrough and subsequently through the analyte capturing membrane.

3. A system according to claim 1 wherein:
the analyte is an inorganic acid;
the analyte capturing membrane incorporates negatively charged groups which bind the inorganic acid;
the eluant comprises a salt which causes the inorganic acid to be released from the membrane and the at least one reagent comprises a reactive agent which reacts with the inorganic acid in a manner that permits detection of the inorganic acid.

4. A system according to claim 3 wherein the analyte is citric acid, and wherein:
the analyte capturing membrane comprises an anionic membrane;
the eluant comprises 0.5 M NaCl; and
the reagent comprises a solution of sulfanilic acid and sodium nitrite which is combined with the eluant after the eluant passes through the analyte capture membrane.

5. A system according to claim 1 wherein
the analyte capture membrane is a first analyte capture membrane, and further comprising:
a second analyte capture membrane located upstream of the first analyte capture membrane such that the sample passes through the second analyte capture membrane before passing through the first analyte capture membrane, the second analyte capture membrane being operative to capture an analyte other than the analyte captured by the first analyte capture membrane.

6. A system according to claim 5, wherein
the second analyte capture membrane is a weakly basic membrane that retains weak inorganic acids; and
the first analyte capture membrane is a strongly basic membrane that retains strong inorganic acids.

7. A system according to claim 6, wherein
the second analyte capture membrane is structured to retain acetic acid; and
the first analyte capture membrane is structured to retain citric acid.

8. A system according to claim 7 wherein
the reagent is a first reagent comprising a solution of sulfanilic acid and sodium nitrite which is combined with the eluant after the eluant passes through the first analyte capture membrane, and further comprising:
a second reagent which comprises xylenol orange in neutralized isopropanol which is combined with the eluant after the eluant passes through the second analyte capture membrane.

9. A system according to claim 1, further comprising
an acidity detector operative to detect the acidity of the sample after the sample has passed through the analyte capture membrane.

10. A system according to claim 2 wherein
the analyte capture membrane is operative to bind proteins present in the sample, and further comprising
a second analyte capture membrane which is operative to bind surfactants present in the sample.

11. A system according to claim 10, wherein
the at least one reagent comprises an enzyme that reacts with lipids to separate glycerol from the free fatty acids of the lipids, and comprises at least one other enzyme that reacts with the separated glycerol to produce a detectable signal.

12. A system according to claim 11 wherein
the at least one reagent comprises glycerol kinase, adenosine triphosphate, glycerol-1 phosphates, aminoantipyrine, and peroxidase.

13. A system according to claim 1 wherein
the analyte capture membrane is operative to bind proteins contained in the sample;
the eluant comprises a salt solution; and
the at least one reagent comprises an indicator solution that produces a detectable signal when mixed with protein.

14. A system according to claim 13, wherein
the at least one reagent comprises a solution containing brilliant blue, methanol, and phosphoric acid.

15. A system according to claim 14 further comprising
a spectrometer operative to detect the presence of a detectable signal at a desired wavelength of light.

16. A system according to claim 1 wherein
the analyte capture membrane is structured to bind nucleic acids contained in the sample;
the eluant comprises a salt solution; and
the at least one reagent comprises at least one nucleic acid primer having a nucleotide sequence that is complementary to at least one nucleotide sequence of the nucleic acids retained by the analyte capture membrane.

17. A system according to claim 16, further comprising
at least one additional analyte capture membrane structured to bind nucleic acids contained in the sample, each of the membranes being serially arranged such that the sample passes through one of the analyte capture membranes before passing through another analyte capture membrane.

18. A system according to claim 16 wherein
the analyte capture membrane is an anionic membrane.

19. A system according to claim 16 wherein
the analyte capture membrane comprises antibodies structured to bind to nucleic acids, and being selective for nucleic acids of a desired microbe.

20. A system according to claim 16 further comprising
a device to perform a polymerase chain reaction using the nucleic acid primers of the at least one reagent and the nucleic acids obtained from the sample.

21. A system according to claim 16, further comprising
a first membrane which is operative to prevent extraneous matter contained in the sample from passing therethrough, while allowing a filtrate containing said analyte to pass therethrough and subsequently through the analyte capturing membrane.

22. A system for determining the presence of an analyte in a matrix which contains matter other than said analyte and an interferant which interferes with the analytical test to be used to determine the presence of the analyte, said system comprising:
at least one interferant-capturing membrane which is operative to capture the interferant while allowing a filtrate which contains the analyte but is substantially devoid of the interferant to pass therethrough; and,
a reagent which is combineable with the filtrate containg the analyte to provide a filtrate/reagent/analyte admixture from which the analyte may be determined.

23. A system according to claim 22 further comprising
a first membrane which is operative to prevent some of the matter of said matrix from passing therethrough, while allowing a filtrate containing the interferant and the analyte to pass therethrough.

24. A system according to 22 for additionally analyzing the interferant, said system further comprising:
an eluant for eluting the interferant from the interferant-capturing membrane so as to provide an eluant/interferant admixture; and
a second reagent that is combinable with the eluant/interferant admixture to provide an eluant/interferant/reagent admixture from which the interferent can be determined.

25. A system according to 22 wherein the analyte is free fatty acid and the interferant is an inorganic acid, and wherein:
the interferant-capturing membrane comprises an anionic membrane which captures inorganic acids; and,
the reagent for determining the presence of the analyte comprises xylenol orange and neutralized isopropanol.

26. A system according to 25 further comprising:
an eluant which is useable to elute the inorganic acid from the interferant-capturing membrane to provide an eluant/inorganic acid admixture; and,
a second reagent that is combinable with the eluant/reagent admixture to facilitate determination of the inorganic acid.

27. A system according to 22 wherein the at least one interferant-capturing membrane comprises:
a first interferant-capturing membrane that is operative to capture a portion of the interferant; and,
a second interferant-capturing membrane that is operative to capture a second portion of the interferant.

28. A system according to claim 27 wherein the first interferant-capturing membrane is operative to capture a weak inorganic acid, and wherein the second interferant-capturing membrane is operative to capture a strong inorganic acid.

29. A system according to claim 28 wherein the first interferant-capturing membrane is operative to capture acetic acid, and wherein the second interferant-capturing membrane is opertative to capture phosphoric acid, and wherein the reagent reacts with free fatty acids filtered from the matrix to produce a detectable signal.

30. A system for detecting the presence of a microbe in a sample, comprising
at least one analyte capture membrane operative to capture nucleic acids contained in the sample;
an eluant for eluting the nucleic acids from the capture membrane so as to provide an eluant/nucleic acid admixture; and
at least one nuclec acid primer structured to hybridize to nucleic acids of microbes suspected of being present in the sample and provided in an amount to facilitate amplification of the nucleic acids that hybridize to the nucleic acid primers so that the nucleic acids of the microbes can be detected.

31. A system according to claim 30, comprising a plurality of analyte capturing membranes.

32. A system according to claim 30, wherein the analyte capturing membrane comprises an anionic membrane.

33. A system according to claim 30, wherein the analyte capturing membrane comprises antibodies attached to the membrane and being operative to bind to the nucleic acids in the sample.

34. A system according to claim 33, wherein the analyte capturing membrane comprises antibodies attached to the membrane and being operative to bind to DNA of a microbe suspected of being present in the sample.

35. A system according to claim 30 wherein the eluant comprises a salt solution.

36. A system according to claim 35 wherein the eluant comprises 1 M NaCl.

37. A system according to claim 30, further comprising a particulate removal membrane positioned with respect to the analyte capture membrane such that the sample passes through the particulate removal membrane to retain particles of the sample, and to pass the nucleic acids in the sample to the analyte capture membrane.

38. A system according to claim 30, further comprising a homogenizer that is operative to homogenize the sample before the sample is applied to the membrane.

39. A system according to claim 30, further comprising a device that is operative to amplify the nucleic acids using a polymerase chain reaction.

40. A system according to claim 30, further comprising a sensor to detect a signal indicating the presence of a nucleic acid of a microbe in the sample.

41. A system according to claim 30, wherein the nucleic acid primers are structured to hybridize to a nucleic acid of e coli bacteria.

42. A system according to claim 41, wherein the nucleic acid primers are structured to hybridize to a nucleic acid of e coli H157.
